(19) United States
(12) Patent Application Publication
(10) Pub. No.: US 2024/0325759 A1
(43) Pub. Date: Oct. 3, 2024

(54) AUTOMATIC DETERMINATION OF STIMULATION AMPLITUDE

(71) Applicant: Leon Mauricio Juarez Paz, Berlin (DE)

(72) Inventor: Leon Mauricio Juarez Paz, Berlin (DE)

(21) Appl. No.: 18/618,726

(22) Filed: Mar. 27, 2024

Related U.S. Application Data
(60) Provisional application No. 63/455,818, filed on Mar. 30, 2023.

(51) Int. Cl. A61N 1/36; A61N 1/372

(52) U.S. Cl. CPC A61N 1/36128; A61N 1/37235

(57) ABSTRACT

This document discusses neurostimulation devices, systems, and methods. A neurostimulation system receives anatomical information associated with a anatomical structure of the patient, receives stimulation settings from previous neurostimulation sessions that includes stimulation location and stimulation amplitude, identifies volume tissue activated features using the anatomical information and the stimulation settings, determines a regression model for the volume of tissue activated features, and determines stimulation amplitude settings for the neurostimulation according to the regression model.

### CLAIM OF PRIORITY

This application claims the benefit of U.S. Provisional Application No. 63/455,818 filed on Mar. 30, 2023, which is hereby incorporated by reference in its entirety.

### TECHNICAL FIELD

This document relates generally to medical devices, and more particularly, to systems, devices, and methods for determining and setting of stimulation parameters for programming an electrical neurostimulation system.

### BACKGROUND

Neurostimulation, also referred to as neuromodulation, has been proposed as a therapy for a number of conditions. Examples of neurostimulation include Deep Brain Stimulation (DBS), Spinal Cord Stimulation (SCS), Peripheral Nerve Stimulation (PNS), and Functional Electrical Stimulation (FES). Implantable neurostimulation systems have been applied to deliver such a therapy. An implantable neurostimulation system may include an implantable neurostimulator, also referred to as an implantable pulse generator (IPG), and one or more implantable leads each including one or more electrodes. The implantable neurostimulator delivers neurostimulation energy through one or more electrodes placed on or near a target site in the nervous system. An external programming device can be used to program the implantable neurostimulator with stimulation parameters controlling the delivery of the neurostimulation energy.

In one example, the neurostimulation energy is delivered in the form of electrical neurostimulation pulses. The delivery is controlled using stimulation parameters that specify spatial (where to stimulate), temporal (when to stimulate), and informational (patterns of pulses directing the nervous system to respond as desired) aspects of a pattern of neurostimulation pulses. Neurostimulation systems may offer many programmable options for the parameters of the neurostimulation to customize the neurostimulation therapy for a specific patient. For some types of neurostimulation (e.g., DBS) the efficacy of the neurostimulation for the patient may depend on an intricate balance of stimulation location coupled with the programmed stimulation waveform. However, the number of programmable options can create an extensive parameter search space for the physician or clinician. Finding the optimal neurostimulation parameters may take a lot of time in the clinic for both the clinic staff and the patient.

### SUMMARY

In DBS, electrical neurostimulation therapy is delivered to implantable electrodes located at certain neurostimulation targets in the brain to treat neurological or neurophysiological disorders. Device-based neurostimulation can include techniques to reduce the parameter search space for the physician when customizing neurostimulation parameters to a particular patient.

Example 1 includes subject matter (such as a machine-implemented method of controlling operation of a neurostimulation system to deliver neurostimulation to tissue of a patient using a stimulation lead that includes electrodes) comprising receiving, by the neurostimulation system, anatomical information associated with an anatomical structure of the patient; receiving stimulation settings from previous neurostimulation sessions that includes stimulation location and stimulation amplitude; identifying a volume of tissue activated (VTA) relative to the anatomical structure using the anatomical information and the stimulation settings; determining a regression model relating the VTA to the anatomical structure; and determining stimulation amplitude settings for the neurostimulation according to the regression model.

In Example 2, the subject matter of Example 1 optionally includes determining principal axes of the anatomical structure using the anatomical information; and determining distance of a VTA radius from the anatomical structure and from the stimulation lead.

In Example 3, the subject matter of Example 2 optionally includes determining the principal axes of the anatomical structure that include length, width, and height of a STN of the patient.

In Example 4, the subject matter of one or any combination of Examples 1-3 optionally includes determining a linear regression model relating the VTA to the anatomical structure.

In Example 5, the subject matter of one or any combination of Examples 1-4 optionally includes determining stimulation amplitude settings for different levels of the stimulation lead.

In Example 6, the subject matter of one or any combination of Examples 1-5 optionally includes presenting, utilizing a user interface of the neurostimulation system, a representation of the anatomical structure, the stimulation lead, and a VTA radius relative to the anatomical structure.

In Example 7, the subject matter of one or any combination of Examples 1-6 optionally includes presenting, utilizing a user interface, the VTA radius for different levels of electrodes of the stimulation lead.

In Example 8, the subject matter of one or any combination of Examples 1-7 optionally includes determining VTA radius limits using the stimulation amplitude settings from the previous neurostimulation sessions.

In Example 9, the subject matter of one or any combination of Examples 1-8 optionally includes receiving anatomical information associated with a subthalamic nucleus (STN) of the patient.

Example 10 includes subject matter (such as a programming device for a neurostimulation system) or can optionally be combined with one or any combination of Examples 1-9 to include such subject matter, comprising a port for receiving anatomical information associated with an anatomical structure of a patient into the programming device and a programming control circuit. The programming control circuit is configured to retrieve stimulation settings from previous neurostimulation sessions, wherein the settings include stimulation location and stimulation amplitude; identify a volume of tissue activated (VTA) relative to the anatomical structure using the anatomical information and the stimulation settings; determine a regression model relating the VTA to the anatomical structure; and determine stimulation amplitude settings for the neurostimulation according to the regression model.

In Example 11, the subject matter of Example 10 optionally includes the port being a communication port configured to receive anatomical information from a separate device, and the anatomical information is associated with a subthalamic nucleus (STN) of the patient.

In Example 12, the subject matter of one or both of Examples 10 and 11 optionally includes the port being a communication port configured to receive anatomical information from a separate device, and the anatomical information includes information of position of a stimulation lead relative to the anatomical structure of the patient. The subject matter optionally includes a programming control circuit configured to determine a shape of the anatomical structure using the anatomical information; and determine distance of the VTA from the anatomical structure and from the stimulation lead.

In Example 13, the subject matter of Example 12 optionally includes a programming control circuit is configured to determine length, width, and height of the anatomical structure using the anatomical information.

In Example 14, the subject matter of one or any combination of Examples 10-13 optionally includes a programming control circuit is configured to determine a linear regression model relating the VTA to the anatomical structure.

In Example 15, the subject matter of one or any combination of Examples 10-14 optionally includes a programming control circuit is configured to determine stimulation amplitude settings for different levels of a stimulation lead having electrodes at multiple levels.

In Example 16, the subject matter of one or any combination of Examples 10-15 optionally includes a user interface operatively coupled to the programming control circuit and a programming control circuit configured to present on the user interface a representation of the anatomical structure, the stimulation lead, and a VTA radius relative to the anatomical structure.

In Example 17, the subject matter of Example 16 optionally includes a programming control circuit configured to present, utilizing the user interface, the VTA radius for different levels of electrodes of the stimulation lead.

In Example 18, the subject matter of one or both of Examples 17 and 17 optionally includes a programming control circuit configured to determine VTA radius limits using the stimulation settings from the previous neurostimulation sessions; and present a representation of the VTA radius limits relative to the anatomical structure on the user interface.

Example 19 can include subject matter (or can optionally be combined with one or any combination of Examples 1-18 to include such subject matter, such as a computer readable storage medium including instructions that when performed by a programming control circuit of a programming device of a neurostimulation system, cause the programming device to perform actions comprising receiving anatomical information associated with an anatomical structure of a patient; retrieving stimulation settings from previous neurostimulation sessions, wherein the settings include at least one of stimulation location or stimulation amplitude; identifying a volume of tissue activated (VTA) relative to the anatomical structure using the anatomical information and the stimulation settings; determining a regression model relating the VTA to the anatomical structure; and determining stimulation amplitude settings for the neurostimulation according to the regression model.

In Example 20, the subject matter of claim 19 optionally includes the computer readable storage medium including instructions that cause the programming device to perform actions including determining a VTA radius for different amplitude settings and for different levels of a stimulation lead having electrodes at multiple levels; and presenting, on a user interface of the programming device, a representation of the anatomical structure, the stimulation lead, and the VTA radius relative to the anatomical structure.

These non-limiting examples can be combined in any permutation or combination. This summary is intended to provide an overview of subject matter of the present patent application. It is not intended to provide an exclusive or exhaustive explanation of the disclosure. The detailed description is included to provide further information about the present patent application. Other aspects of the disclosure will be apparent to persons skilled in the art upon reading and understanding the following detailed description and viewing the drawings that form a part thereof, each of which are not to be taken in a limiting sense.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, various embodiments discussed in the present document.

FIG. 1 is an illustration of portions of an example of a neurostimulation system.

FIG. 2 is an illustration of portions of another example of a neurostimulation system.

FIG. 3 is an illustration of an example of an implantable pulse generator (IPG) and an implantable lead system.

FIG. 4 is an illustration of another example of an IPG and an implantable lead system.

FIG. 5 is an illustration of an example of a programming device of a neurostimulation system.

FIG. 6 is an illustration of an implanted Deep Brain Stimulation (DBS) stimulation lead.

FIGS. 7A-7G are illustrations of an example of electrodes of a stimulation lead.

FIG. 8 is a flow diagram of a method to operate a neurostimulation system.

FIG. 9 is an example of a graph including a representation of a subthalamic nucleus (STN) and a stimulation lead.

FIG. 10 is an example of a plot of radius of volume of tissue activated versus anatomical features in relation to a stimulation lead.

FIG. 11 is another example of a graph including a representation of an STN and a stimulation lead.

FIG. 12 is an illustration of a representation of neurostimulation in a two-dimensional space.

### DETAILED DESCRIPTION

In the following detailed description, reference is made to the accompanying drawings which form a part hereof, and in which is shown by way of illustration specific embodiments in which the invention may be practiced. These embodiments are described in sufficient detail to enable those skilled in the art to practice the invention, and it is to be understood that the embodiments may be combined, or that other embodiments may be utilized, and that structural, logical, and electrical changes may be made without departing from the spirit and scope of the present invention. References to “an”, “one”, or “various” embodiments in this disclosure are not necessarily to the same embodiment, and such references contemplate more than one embodiment. The following detailed description provides examples, and the scope of the present invention is defined by the appended claims and their legal equivalents.

This document discusses devices, systems and methods for programming and delivering electrical neurostimulation to a patient or subject. Advancements in neuroscience and neurostimulation research have led to a demand for delivering complex patterns of neurostimulation energy for various types of therapies. The present system may be implemented using a combination of hardware and software designed to apply any neurostimulation (neuromodulation) therapy, including but not being limited to DBS therapy.

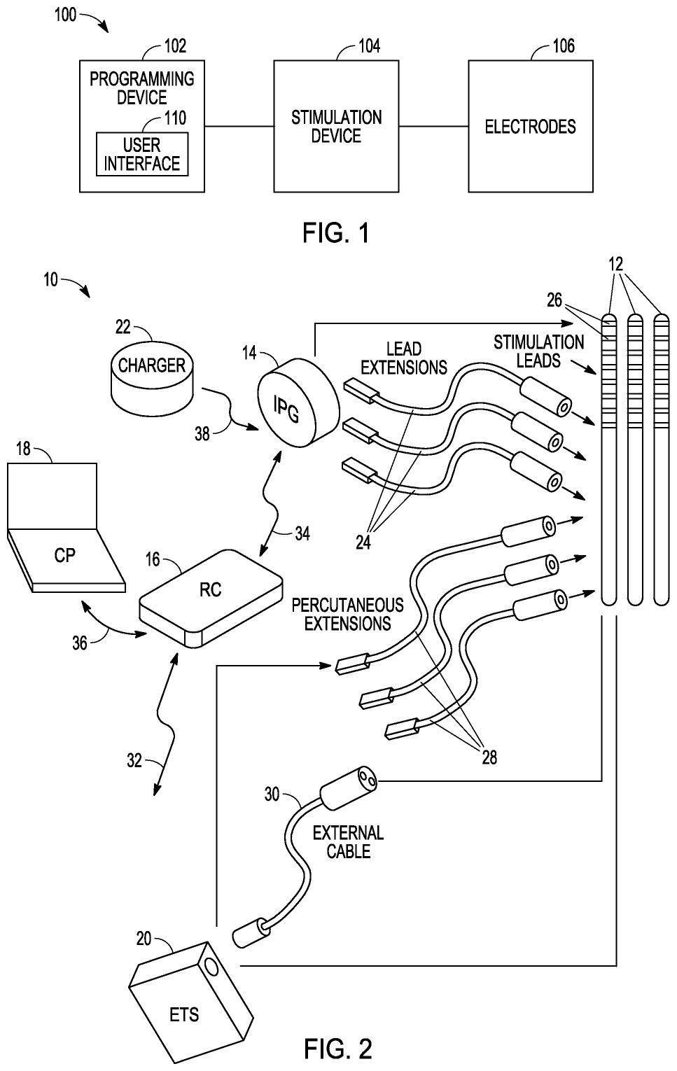

FIG. 1 illustrates an example of portions of a neurostimulation system 100. System 100 includes electrodes 106, a stimulation device 104, and a programming device 102. Electrodes 106 are configured to be placed on or near one or more neural targets in a patient. Stimulation device 104 is configured to be electrically connected to electrodes 106 and deliver neurostimulation energy, such as in the form of electrical pulses, to the one or more neural targets though electrodes 106. The delivery of the neurostimulation is controlled by using multiple stimulation parameters, such as stimulation parameters specifying a pattern of the electrical pulses and a selection of electrodes through which each of the electrical pulses is delivered. In various embodiments, at least some of the stimulation parameters are programmable by a user, such as a physician or other caregiver who treats the patient using system 100. Programming device 102 provides the user with accessibility to the user-programmable parameters. In various embodiments, programming device 102 is configured to be communicatively coupled to stimulation device 104 via a wired or wireless link.

In this document, a “user” includes a physician or other clinician or caregiver who treats the patient using system 100; a “patient” includes a person who receives or is intended to receive neurostimulation delivered using system 100. In various embodiments, the patient can be allowed to adjust his or her treatment using system 100 to certain extent, such as by adjusting certain therapy parameters and entering feedback and clinical effect information.

In various embodiments, programming device 102 can include a user interface 110 that allows the user to control the operation of system 100 and monitor the performance of system 100 as well as conditions of the patient including responses to the delivery of the neurostimulation. The user can control the operation of system 100 by setting and/or adjusting values of the user-programmable parameters.

In various embodiments, user interface 110 can include a graphical user interface (GUI) that allows the user to set and/or adjust the values of the user-programmable parameters by creating and/or editing graphical representations of various stimulation waveforms. Such waveforms may include, for example, a waveform representing a pattern of neurostimulation pulses to be delivered to the patient as well as individual waveforms that are used as building blocks of the pattern of neurostimulation pulses, such as the waveform of each pulse in the pattern of neurostimulation pulses. The GUI may also allow the user to set and/or adjust stimulation fields each defined by a set of electrodes through which one or more neurostimulation pulses represented by a waveform are delivered to the patient. The stimulation fields may each be further defined by the distribution of the current of each neurostimulation pulse in the waveform. In various embodiments, neurostimulation pulses for a stimulation period (such as the duration of a therapy session) may be delivered to multiple stimulation fields.

In various embodiments, system 100 can be configured for neurostimulation applications. User interface 110 can be configured to allow the user to control the operation of system 100 for neurostimulation. For example, system 100 as well as user interface 110 can be configured for DBS applications. The DBS configurations include various features that may simplify the task of the user in programming the stimulation device 104 for delivering DBS to the patient, such as the features discussed in this document.

FIG. 2 is an illustration of portions of another example of a neurostimulation system 10 that includes one or more stimulation leads 12 and an implantable pulse generator (IPG) 14. The system 10 can also include one or more of an external remote control (RC) 16, a clinician's programmer (CP) 18, an external trial stimulator (ETS) 20, or an external charger 22. The IPG 14 can optionally be physically connected via one or more lead extensions 24, to the stimulation lead(s) 12. Each lead carries multiple electrodes 26 arranged in an array. The IPG 14 includes pulse generation circuitry that delivers electrical stimulation energy in the form of, for example, a pulsed electrical waveform (i.e., a temporal series of electrical pulses) to the electrode array 26 in accordance with a set of stimulation parameters. The IPG 14 can be implanted into a patient's body, for example, below the patient's clavicle area or within the patient's buttocks or abdominal cavity. The implantable pulse generator can have multiple stimulation channels (e.g., 8, 16, or 32) which may be independently programmable to control the magnitude of the current stimulus from each channel. The IPG 14 can have one, two, three, four, or more connector ports, for receiving the terminals of the leads 12.

The ETS 20 may also be physically connected, optionally via the percutaneous lead extensions 28 and external cable 30, to the stimulation leads 12.

The ETS 20, which may have similar pulse generation circuitry as the IPG 14, can also deliver electrical stimulation energy in the form of, for example, a pulsed electrical waveform to the electrode array 26 in accordance with a set of stimulation parameters. One difference between the ETS 20 and the IPG 14 is that the ETS 20 is often a non-implantable device that is used on a trial basis after the neurostimulation leads 12 have been implanted and prior to implantation of the IPG 14, to test the responsiveness of the stimulation that is to be provided. Any functions described herein with respect to the IPG 14 can likewise be performed with respect to the ETS 20.

The RC 16 may be used to telemetrically communicate with or control the IPG 14 or ETS 20 via a wireless communications link 32. Once the IPG 14 and neurostimulation leads 12 are implanted, the RC 16 may be used to telemetrically communicate with or control the IPG 14 via communications link 34. The communication or control allows the IPG 14 to be turned on or off and to be programmed with different stimulation parameter sets. The IPG 14 may also be operated to modify the programmed stimulation parameters to actively control the characteristics of the electrical stimulation energy output by the IPG 14. The CP 18 allows a user, such as a clinician, the ability to program stimulation parameters for the IPG 14 and ETS 20 in the operating room and in follow-up sessions. The CP 18 may perform this function by indirectly communicating with the IPG 14 or ETS 20, through the RC 16, via a wireless communications link 36. Alternatively, the CP 18 may directly communicate with the IPG 14 or ETS 20 via a wireless communications link (not shown). The stimulation parameters provided by the CP 18 are also used to program the RC 16, so that the stimulation parameters can be subsequently modified by operation of the RC 16 in a stand-alone mode (i.e., without the assistance of the CP 18).

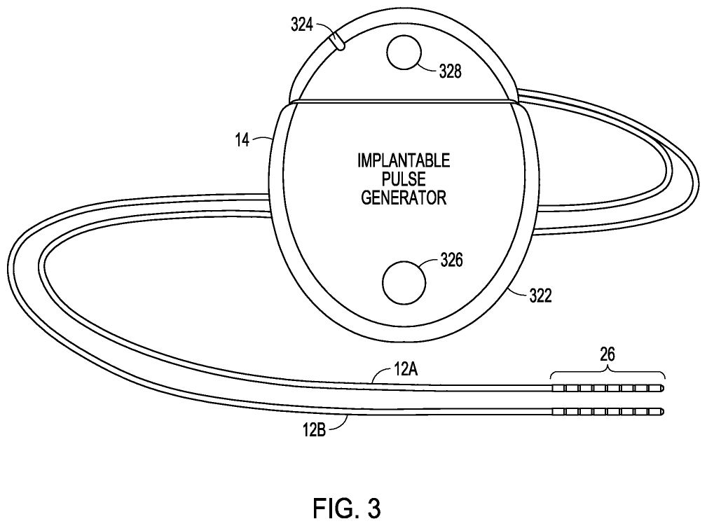

FIG. 3 is an illustration of an example of an IPG 14 (e.g., IPG 14 in FIG. 2) and an implantable lead system that includes stimulation leads (e.g., stimulation leads 12 in FIG. 2). The IPG 14 can be used as stimulation device 104 in FIG. 1. As illustrated in FIG. 3, IPG 14 that can be coupled to implantable leads 12A and 12B at a proximal end of each lead. The distal end of each lead includes electrical contacts or electrodes 26 for contacting a tissue site targeted for electrical neurostimulation. As illustrated in FIG. 3, leads 12A and 12B each include 8 electrodes 26 at the distal end. The number and arrangement of leads 12A and 12B and electrodes 26 as shown in FIGS. 2 and 3 are only examples, and other numbers and arrangements are possible. In various examples, the lead electrodes 26 are ring electrodes. In various examples the lead electrodes 26 include one or more segmented electrodes.

The IPG 14 can include a hermetically sealed IPG case 322 to house the electronic circuitry of IPG 14. IPG 14 can include an electrode 326 formed on IPG case 322. IPG 14 can include an IPG header 324 for coupling the proximal ends of leads 12A and 12B. IPG header 324 may optionally also include an electrode 328. One or both of electrodes 326 and 328 may be used as a reference electrode.

The implantable leads and electrodes may be configured by shape and size to provide electrical neurostimulation energy to a neuronal target included in the subject's brain. Neurostimulation energy can be delivered in a monopolar (also referred to as unipolar) mode using an IPG electrode and one or more electrodes selected from electrodes 26. Neurostimulation energy can be delivered in a bipolar mode using a pair of electrodes of the same lead (lead 12A or lead 12B).

Neurostimulation energy can be delivered in an extended bipolar mode using one or more electrodes of a lead (e.g., one or more electrodes of lead 12A) and one or more electrodes of a different lead (e.g., one or more electrodes of lead 12B).

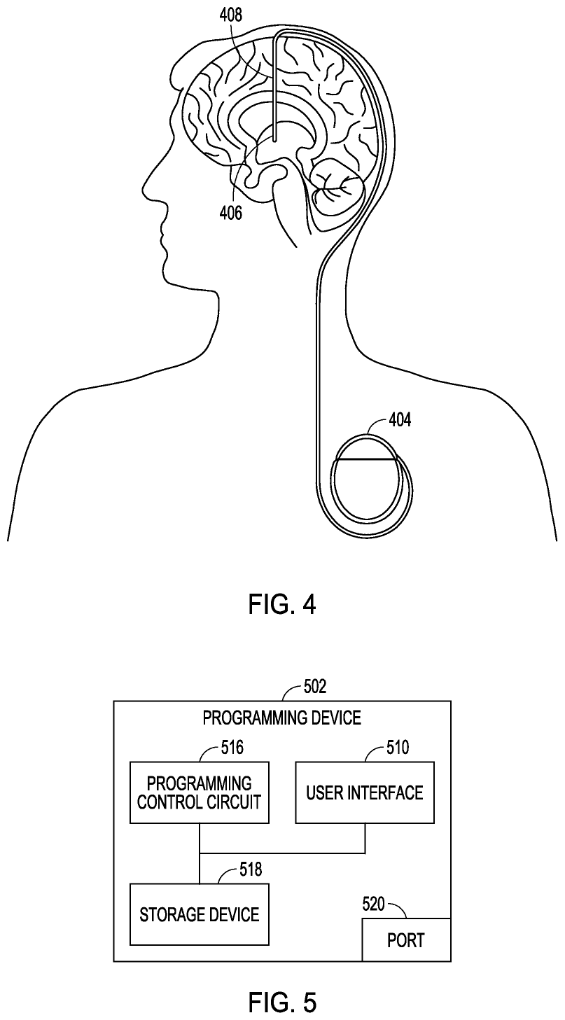

FIG. 4 illustrates another example of an IPG 404 and an implantable lead system 408 arranged to provide neurostimulation to a patient. An example of IPG 404 includes IPG 14 of FIGS. 2 and 3. An example of lead system 408 includes one or more of leads 12A and 12B in FIG. 3. The lead distal end 406 is implanted near a stimulation target. In the illustrated embodiment, implantable lead system 408 is arranged to provide Deep Brain Stimulation (DBS) to a patient, with the stimulation target being neuronal tissue in a subdivision of the thalamus of the patient's brain. Other examples of DBS targets include neuronal tissue of the globus pallidus (GPi), the subthalamic nucleus (STN), the pedunculopontine nucleus (PPN), substantia nigra pars reticulate (SNr), cortex, globus pallidus externus (GPe), medial forebrain bundle (MFB), periaquaductal gray (PAG), periventricular gray (PVG), habenula, subgenual cingulate, ventral intermediate nucleus (VIM), anterior nucleus (AN), other nuclei of the thalamus, zona incerta, ventral capsule, ventral striatum, nucleus accumbens, and any white matter tracts connecting these and other structures.

Returning to FIG. 3, the electronic circuitry of IPG 14 can include a stimulation control circuit that controls delivery of the neurostimulation energy. The stimulation control circuit can include a microprocessor, a digital signal processor, application specific integrated circuit (ASIC), or other type of processor, interpreting or executing instructions included in software or firmware. The neurostimulation energy can be delivered according to specified (e.g., programmed) modulation parameters. Examples of setting modulation parameters can include, among other things, selecting the electrodes or electrode combinations used in the stimulation, configuring an electrode or electrodes as the anode or the cathode for the stimulation, specifying the percentage of the neurostimulation provided by an electrode or electrode combination, and specifying stimulation pulse parameters. Examples of pulse parameters include, among other things, the amplitude of a pulse (specified in current or voltage), pulse duration (e.g., in microseconds), pulse rate (e.g., in pulses per second), and parameters associated with a pulse train or pattern such as burst rate (e.g., an “on” modulation time followed by an “off” modulation time), amplitudes of pulses in the pulse train, polarity of the pulses, etc.

FIG. 5 is an illustration of an example of a programming device 502, such as programming device 102 of neurostimulation system 100 in FIG. 1. Programming device 502 includes a storage device 518, a programming control circuit 516, a user interface 510, and a port 520 (e.g., a communication port or Comm Port). Programming control circuit 516 may be implemented using an application-specific integrated circuit (ASIC) constructed to perform one or more particular functions or a general-purpose circuit programmed to perform such functions. A general-purpose circuit can include, among other things, a microprocessor or a portion thereof, a microcontroller or portions thereof, and a programmable logic circuit or a portion thereof. The storage device 518 may be a memory integral to the programming control circuit 516, or a separate memory device. The programming device 502 may receive information from a separate device via the port 520. The information may be stored in the storage device 518 or used by the programming control circuit 516 without first being stored in the storage device 518.

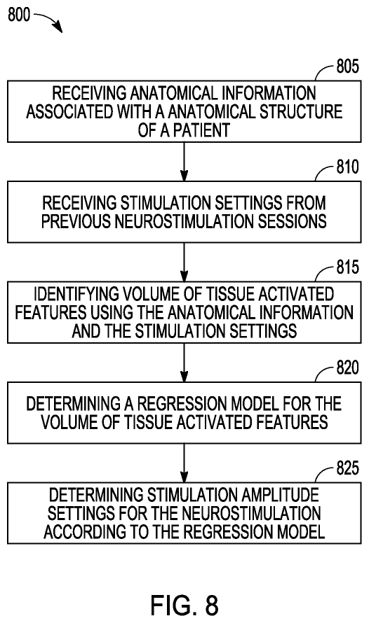

Programming control circuit 516 generates the various stimulation parameters that control the delivery of the neurostimulation pulses according to a specified stimulation configuration that can define, for example, stimulation waveform and electrode configuration. User interface 510 represents an embodiment of user interface 110 in FIG. 1. Storage device 518 stores information used by programming control circuit 516, such as information about a stimulation device (e.g., stimulation device 104 in FIG. 1) that relates the stimulation configuration to the stimulation parameters, and information relating the stimulation configuration to a volume of tissue activation in the patient. In various embodiments, programming control circuit 516 can be configured to support one or more functions allowing for programming of stimulation devices, such as stimulation device 104 including its various embodiments as discussed in this document and determine stimulation amplitude or radius as described in regard to FIGS. 8-11.

In various embodiments, user interface 510 can allow for definition of a pattern of neurostimulation pulses for delivery during a neurostimulation therapy session by creating and/or adjusting one or more stimulation waveforms using a graphical method. The definition can also include definition of one or more stimulation fields each associated with one or more pulses in the pattern of neurostimulation pulses. As used in this document, a “stimulation configuration” can include the pattern of neurostimulation pulses including the one or more stimulation fields, or at least various aspects or parameters of the pattern of neurostimulation pulses including the one or more stimulation fields. Stimulation configuration can include the electrode configuration used to provide the electrical stimulation. In various embodiments, user interface 510 includes a GUI that allows the user to define the pattern of neurostimulation pulses and perform other functions using graphical methods. In this document, “neurostimulation programming” can include the definition of the one or more stimulation waveforms, including the definition of one or more stimulation fields.

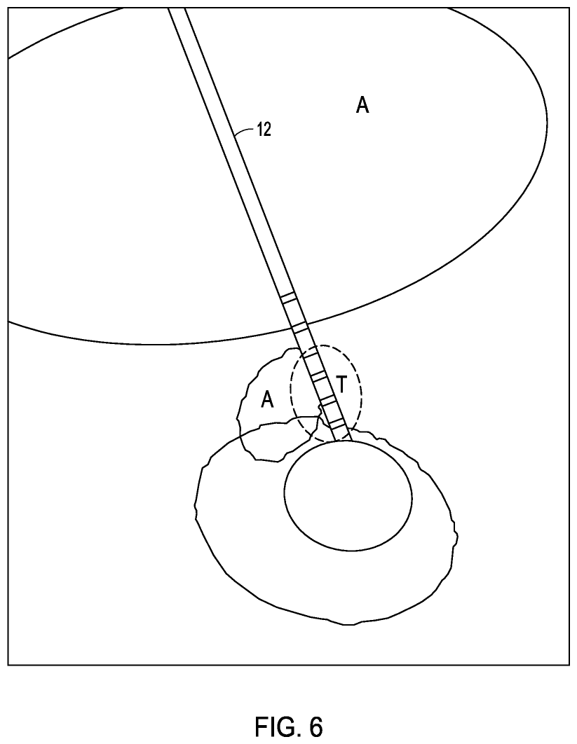

FIG. 6 is an image of an implanted DBS stimulation lead 12 (e.g., stimulation lead 12 of FIG. 2). The image in the example shows the physiological target structure (T) for the neurostimulation and avoidance structures (A) that the physician would like to avoid activating with the neurostimulation. For example, the physiological target structure may be the motor subthalamic nucleus (STN) and the avoidance structures may be the nonmotor STN and internal capsule (capsula interna).

The stimulation lead 12 includes multiple electrodes and the electrodes can be configured into multiple electrode configurations. Different electrode configurations can steer the neurostimulation energy (e.g., electrical current) toward different volumes of tissue. Additionally, the electrode configuration can include “fractionalization” of the electrical current flowing through the selected one or more electrodes. In fractionalization, a fraction of an overall pulse amplitude is assigned to each of the electrodes included in the electrode configuration.

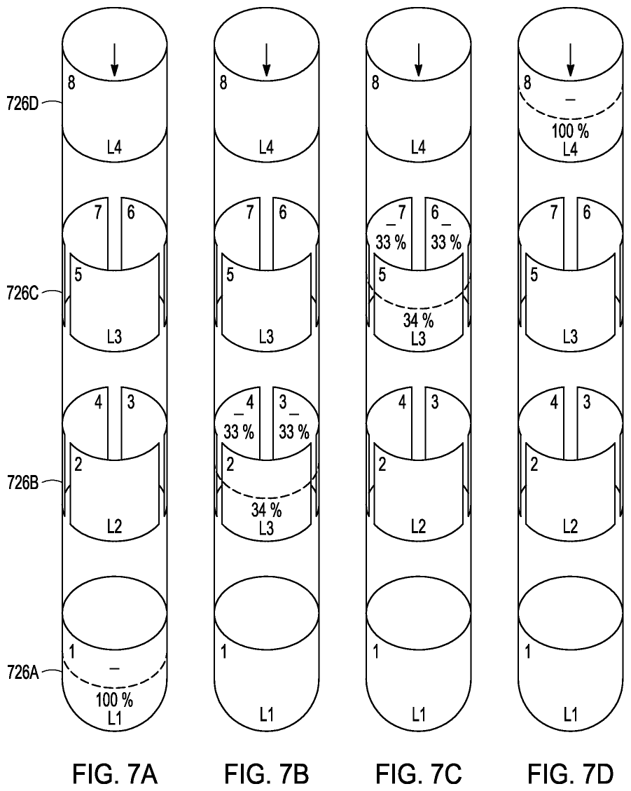

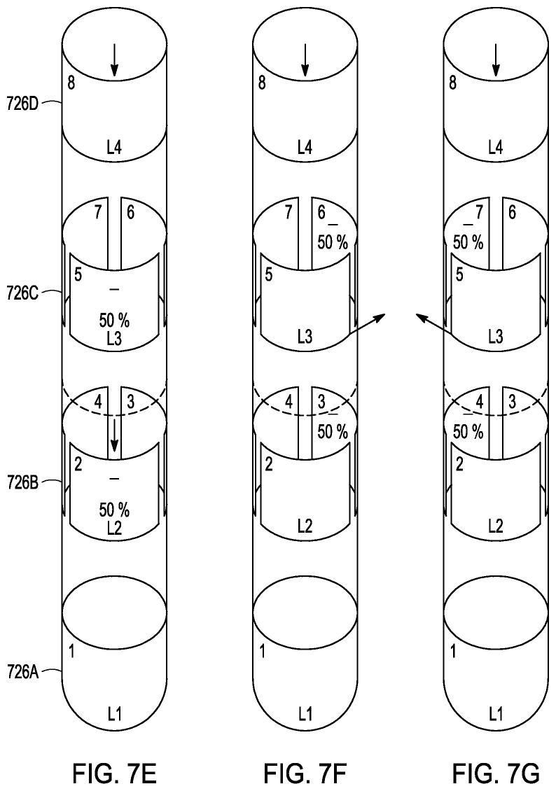

FIGS. 7A-7G are illustrations of an example of electrodes of a stimulation lead (e.g., stimulation lead 12 of FIG. 6). The electrodes are at four levels of the lead labeled L1-L4. At L1, the lead includes an electrode 726A that is a tip electrode. At levels L2 and L3, the electrodes are segmented electrodes 726B, 726C that include three electrode segments. In variations, the segmented electrodes 726B, 726C can have two segments or four segments, or segmented electrode 726B can have a different number of segments than electrode 726C. At level L4, the lead includes a ring electrode 726D. Any combination of the electrodes and electrode segments can be included in an electrode configuration, and the current delivered can be divided among the electrodes and electrode segments.

In the examples of FIGS. 7A-7D, different electrodes are highlighted to show how the current can be delivered to different levels or heights of the electrode corresponding to different locations of the target region Tis FIG. 6. Current delivered to tip electrode 726A can also emanate downward into the tissue.

In the examples of FIGS. 7E-7G, electrode configurations are shown that use segmented electrodes 726B, 726C. Segments of the electrodes are highlighted to show how current can be delivered to multiple levels and in a particular direction using electrode segments. In FIG. 7E, current is delivered to levels L2 and L4 and to the electrode segments at the front of segmented electrodes 726B, 726C. Two electrode segments are included in the electrode configuration of FIG. 7E. The current may be evenly divided among the electrode segments or different fractions of current can be provided to the electrode segments. In FIG. 7F, current is delivered to levels L2 and L4 and to the electrode segments at the right and slightly toward the rear of segmented electrodes 726B, 726C. In FIG. 7G, current is delivered to levels L2 and L4 and to the electrode segments at the left and slightly toward the rear of segmented electrodes 726B, 726C. The examples in FIGS. 7A-7G show how current can be steered by the programming control circuit 516 of FIG. 5 toward different tissue volumes that are selected as the target volume when the stimulation lead is implanted.

As DBS systems become more complex, the number of programmable options available can become large. Neurostimulation systems can be programmable in stimulation sites, stimulation electrode combinations, stimulation pulse amplitude, pulse width, pulse rate, and pulse pattern to provide many different neurostimulation waveforms. This produces a large parameter space that needs to be searched to find the best stimulation configuration solution for the patient.

In one approach to finding the stimulation settings appropriate for a particular patient, the physician performs a monopolar review of settings available on a neurostimulation device. In monopolar review, the physician activates one electrode at a time and increases the stimulation amplitude. While making the changes, the physician asks the patient for feedback of the settings, or one more sensors can be used to detect the results of the stimulation and provide the feedback.

The physician may increase the stimulation amplitude until the patient feels a side effect such as dysarthria. The physician then stops increasing the amplitude and sets the amplitude to setting below the amplitude that caused the side effect. The physician may also find the lowest amplitude setting that provide benefit to the patient. The difference between the high side effect threshold and the lower benefit threshold can be referred to as a therapeutic window. This can be performed by the physician for all electrodes and electrode segments of the stimulation lead. The physician may settle on a stimulation configuration solution with the stimulation settings resulting in the largest therapeutic window for the patient. It can be seen that this process is time consuming for the physician and patient. The process may have to be repeated if the neurostimulation therapy needs adjusting at a later date.

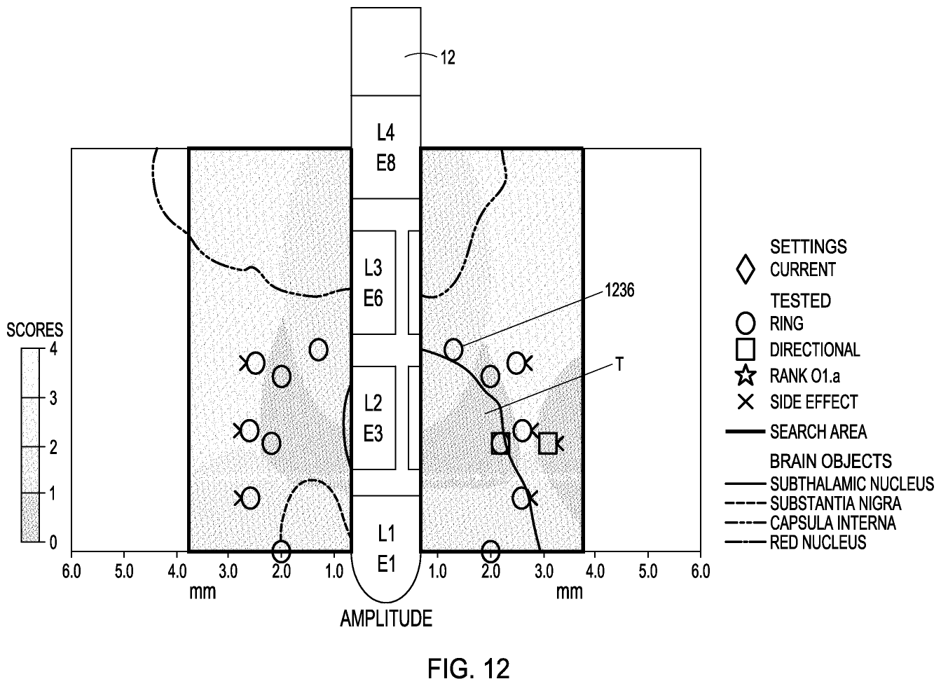

Another approach is to search neurostimulation settings in a two-dimensional space. For example, one dimension of the space may be the stimulation amplitude and the other dimension can be the location of the stimulation (as determined by one or both of electrode level and electrode segment). FIG. 12 is an illustration of a two-dimensional mapping. The vertical axis is the electrode position, and the horizontal axis is the stimulation amplitude. The mapping shows tested points 1236 and can include a representation of the stimulation target (T). The two-dimensional mapping can be constructed to aid the physician in visualizing the neurostimulation settings and variations in the benefit of the neurostimulation therapy to help the physician arrive at a stimulation configuration solution.

Both the monopolar approach and the two-dimensional approach are “naïve” approaches that begin from scratch without any record of stimulation settings that were seen to be good for a patient. For both approaches, many iterations may be needed to find stimulation settings that are beneficial for the patient.

FIG. 8 is a flow diagram of a method 800 to operate a neurostimulation system (e.g., the neurostimulation system 100 of FIG. 1) to automatically determine a stimulation configuration solution for the neurostimulation system. The programming device (e.g., programming device 102 of FIG. 1, or programming device 502 of FIG. 5) of the neurostimulation system uses anatomical information of the patient being treated and previous stimulation settings relevant to the patient to automatically determine stimulation amplitude settings for the patient. This reduces the amount of time needed to find a stimulation configuration solution for the patient.

At block 805, the neurostimulation system receives anatomical information associated with a anatomical structure of the patient. The anatomical structure may be the target or may include the target for the neurostimulation. The anatomical information can be received into the programming device of the neurostimulation system via a communication port of the programming device. The anatomical information can include information regarding the position of an implanted stimulation lead relative to the anatomical structure. For example, the anatomical information can include imaging information, such as information related to the imaging in FIG. 6, where the anatomical structure is the STN of the patient. The position of electrodes relative to the STN can be extracted from the imaging information. The position information can be extracted by the programming device or by a separate device providing the anatomical information.

At block 810, the neurostimulation system receives stimulation settings from previous neurostimulation sessions. The stimulation settings may be preloaded into a storage device of the system (e.g., storage device 518 of programming device 502 in FIG. 5) or the neurostimulation system may download the stimulation settings from a cloud server when searching the available stimulation parameter space for settings. The stimulation settings include stimulation locations and stimulation amplitudes. The stimulation settings may correspond to points of one or more two-dimensional mappings. The stimulation settings may correspond to one or more stimulation configuration solutions.

At block 815, the programming device identifies a volume of tissue activated (VTA) and the location of the VTA relative to the anatomical structure using the anatomical information and the stimulation settings. Stimulation amplitude is related to the radius of the volume of tissue activated (i.e., the VTA radius). The electrode configuration of the previous stimulation sessions and the stimulation amplitude can be used to determine the VTA of the previous neurostimulation sessions. The programming device can compare the previous session VTAs and the current anatomical information (e.g., one or more of the target anatomical structure, any avoidance structures, and the current lead position) to determine the stimulation settings relevant for the current patient.

The programming device may determine the shape of the anatomical structure. For example, the programming control circuit of the device may determine the principal axes (e.g., length, width, and height) of the STN in FIG. 6 using the anatomical information. Imaging information for the anatomical structure may be obtained by a magnetic resonance imaging system or a tomography system. The imaging information may be provided to a separate device to extract the distance from the stimulation lead to one or more of the anterior border of the anatomical structure, the posterior border of the anatomical structure, the lateral border of the anatomical structure, the medial border of the anatomical structure, and the center of the anatomical structure. The programming control circuit may receive the distance information from the separate device and calculate the length, width, and height of the principal structure using the distance information.

Based on the shape of the target anatomical structure, the programming device may identify anatomical features (e.g., anterior border, posterior border, etc.) that would be activated by a VTA from the previous sessions. In certain examples, the programming device also compares the condition or disease of the patient to the patient condition of the previous neurostimulation sessions to identify relevant stimulation settings.

At block 820, the programming device determines a regression model relating the identified VTA to the anatomical structure. For instance, the regression model may relate the VTA radius and height to the anatomical features activated by the VTA. At block 825, the programming device uses the regression model to determine the stimulation amplitude setting at different locations along an axis of the anatomical structure. The stimulation amplitude settings may be suggested amplitude settings recommended to the clinician. In some examples, the stimulation amplitude settings are presented to the clinician (e.g., in a display of a user interface), and the clinician either accepts or modifies the suggested amplitude settings.

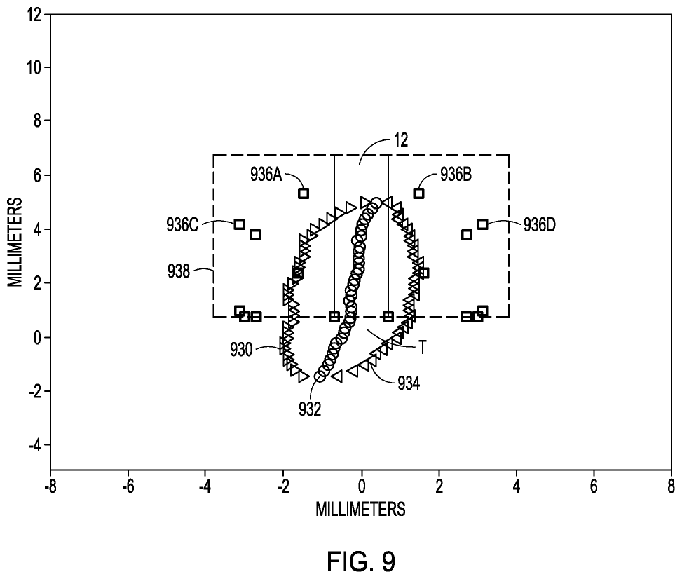

FIG. 9 is an example of a graph including a representation of an STN (T) of a patient and a stimulation lead 12. The representation of the STN includes points of the medial border 930, center 932, and lateral border 934 of the STN. The points and the position of the stimulation lead 12 may have been extracted from image data using a separate device. The box 938 in the graph may represent a boundary of the tissue volume activated in the previous neurostimulation settings. The small squares in the graph correspond to stimulation settings of one or more previous neurostimulation sessions that produced good results. Each of the small squares corresponds to a VTA radius from the stimulation lead 12 and a height relative to the stimulation lead 12. The VTA radii are generated by applying neurostimulation of a specific stimulation amplitude to ring electrodes of the stimulation lead 12, and the position of the squares are symmetrical or mirrored about the stimulation lead 12. For instance, small squares 936A, 936B are the same VTA radius generated using an electrode configuration of the stimulation lead 12, and small squares 936C, 936D are the same stimulation radius.

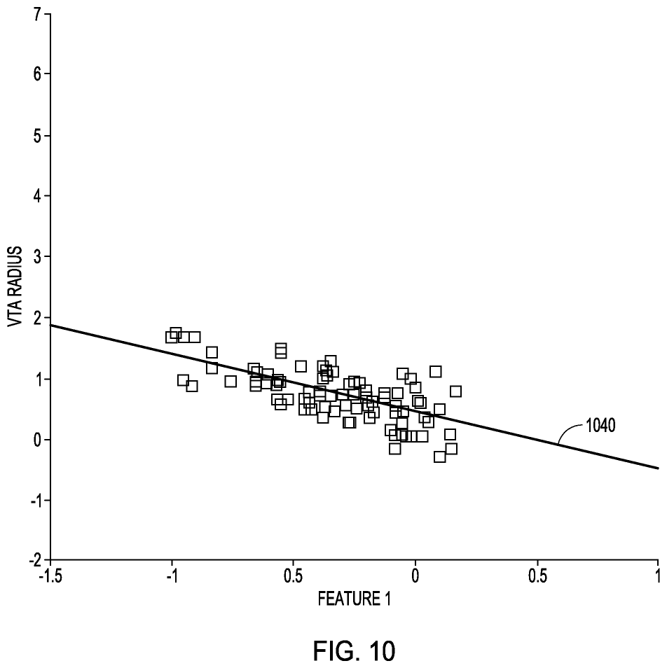

FIG. 10 is a plot of the VTA radius versus anatomical features for the stimulation settings in FIG. 9. The horizontal axis is the distance of the small squares in FIG. 9 to anatomical features such as the center of the lead, the center of the STN, the medial border of the STN, and the lateral border of the STN. The vertical axis is the VTA radius related to the features. If it is known how far away the VTA radius needs to be from the lead, the stimulation amplitude to be applied to the lead can be determined. The points on the graph show how far away from the lead the stimulation needs to reach to get a good result.

FIG. 10 shows a regression model 1040 that correlates the VTA radius and the anatomical features. In the example of FIG. 10, the best fit for the data is a linear regression. The regression model 1040 can be an equation used by the programming device to calculate the stimulation amplitude (using the radius value of the vertical axis) based on the feature distance (the value of the horizontal axis). This allows the programming device to automatically set or to suggest stimulation amplitude settings to the physician, which automatically reduces the parameter space for searching.

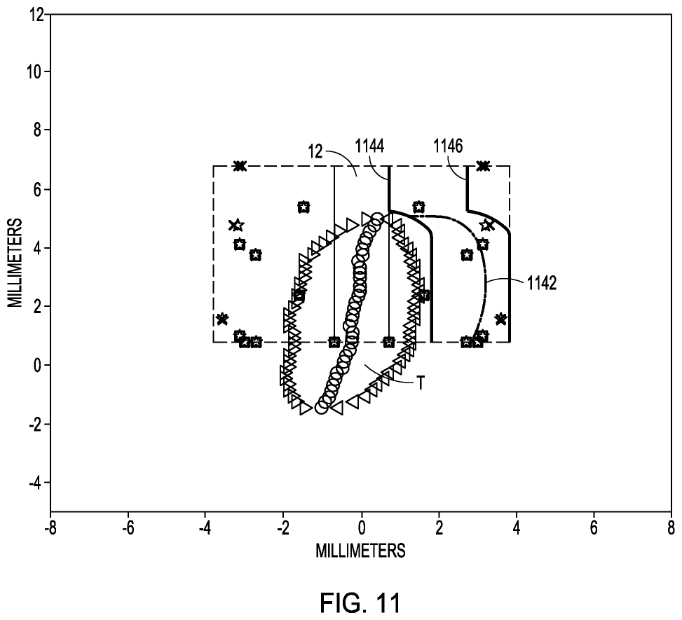

FIG. 11 is another example of a graph including a representation of an STN (T) of a patient and a stimulation lead 12. The graph includes a plot 1142 of the suggested VTA radius as the level of the stimulation lead 12 changes. The graph in FIG. 11 can be displayed on the user interface of the programming device. This may aid the physician in visualizing the VTA resulting from the calculated stimulation settings. The plot 1142 shows the VTA radius changing with the height of the stimulation lead 12 as different levels of electrodes are used for the stimulation. The programming device may iteratively test the VTA radii at different positions along the lead to find the optimal VTA simulation amplitude settings.

The graph in the example of FIG. 11 also shows plots of suggested VTA radius limits including a lower radius limit 1144 and an upper radius limit 1146. The radius limits may be determined statistically from the previous neurostimulation sessions. The physician may choose the stimulation amplitude using the suggested VTA radius and the VTA radius limits.

The devices, systems and methods described herein provide techniques for neuroanatomy-based searching to automatically find optimized deep brain stimulation amplitude for a particular patient quickly. These techniques will continue to be useful as the search space for the optimized stimulation continues to grow with advances in DBS therapy.

The embodiments described herein can be methods that are machine or computer-implemented at least in part. Some embodiments may include a computer-readable medium or machine-readable medium encoded with instructions operable to configure an electronic device or system to perform methods as described in the above examples. An implementation of such methods can include code, such as microcode, assembly language code, a higher-level language code, or the like. Such code can include computer readable instructions for performing various methods. The code can form portions of computer program products. Further, the code can be tangibly stored on one or more volatile or non-volatile computer-readable media during execution or at other times. Various embodiments are illustrated in the figures above. One or more features from one or more of these embodiments may be combined to form other embodiments.

The above detailed description is intended to be illustrative, and not restrictive. The scope of the disclosure should, therefore, be determined with references to the appended claims, along with the full scope of equivalents to which such claims are entitled.

What is claimed is:

1. A method of controlling operation of a neurostimulation system to deliver neurostimulation to tissue of a patient using a stimulation lead that includes electrodes, the method comprising:
receiving, by the neurostimulation system, anatomical information associated with an anatomical structure of the patient;
receiving stimulation settings from previous neurostimulation sessions that includes stimulation location and stimulation amplitude;
identifying a volume of tissue activated (VTA) relative to the anatomical structure using the anatomical information and the stimulation settings;
determining a regression model relating the VTA to the anatomical structure; and
determining stimulation amplitude settings for the neurostimulation according to the regression model.

2. The method of claim 1, wherein the identifying the VTA relative to the anatomical structure includes:
determining principal axes of the anatomical structure using the anatomical information; and
determining distance of a VTA radius from the anatomical structure and from the stimulation lead.

3. The method of claim 2, wherein the determining the principal axes of the anatomical structure includes determining length, width, and height of a STN of the patient.

4. The method of claim 1, wherein determining the regression model includes determining a linear regression model relating the VTA to the anatomical structure.

5. The method of claim 1, wherein the determining the stimulation amplitude settings includes determining stimulation amplitude settings for different levels of the stimulation lead.

6. The method of claim 1, including presenting, utilizing a user interface of the neurostimulation system, a representation of the anatomical structure, the stimulation lead, and a VTA radius relative to the anatomical structure.

7. The method of claim 6, including presenting, utilizing the user interface, the VTA radius for different levels of electrodes of the stimulation lead.

8. The method of claim 6, including:
determining VTA radius limits using the stimulation amplitude settings from the previous neurostimulation sessions; and
presenting, utilizing the user interface, the VTA radius limits relative to the anatomical structure.

9. The method of claim 1, wherein the receiving the anatomical information includes receiving anatomical information associated with a subthalamic nucleus (STN) of the patient.

10. A programming device for a neurostimulation system, the device comprising:
a port for receiving anatomical information associated with an anatomical structure of a patient into the programming device; and
a programming control circuit configured to:
retrieve stimulation settings from previous neurostimulation sessions, wherein the settings include stimulation location and stimulation amplitude;
identify a volume of tissue activated (VTA) relative to the anatomical structure using the anatomical information and the stimulation settings;
determine a regression model relating the VTA to the anatomical structure; and
determine stimulation amplitude settings for the neurostimulation according to the regression model.

11. The device of claim 10, wherein the port is a communication port configured to receive anatomical information from a separate device, and the anatomical information is associated with a subthalamic nucleus (STN) of the patient.

12. The device of claim 10,
wherein the port is a communication port configured to receive anatomical information from a separate device, and the anatomical information includes information of position of a stimulation lead relative to the anatomical structure of the patient; and
wherein the programming control circuit is configured to:
determine a shape of the anatomical structure using the anatomical information; and
determine distance of the VTA from the anatomical structure and from the stimulation lead.

13. The device of claim 12, wherein the programming control circuit is configured to determine length, width, and height of the anatomical structure using the anatomical information.

14. The device of claim 10, wherein the programming control circuit is configured to determine a linear regression model relating the VTA to the anatomical structure.

15. The device of claim 10, wherein the programming control circuit is configured to determine stimulation amplitude settings for different levels of a stimulation lead having electrodes at multiple levels.

16. The device of claim 10, including:
a user interface operatively coupled to the programming control circuit; and
wherein the programming control circuit is configured to present on the user interface a representation of the anatomical structure, the stimulation lead, and a VTA radius relative to the anatomical structure.

17. The device of claim 16, wherein the programming control circuit is configured to present, utilizing the user interface, the VTA radius for different levels of electrodes of the stimulation lead.

18. The device of claim 16, wherein the programming control circuit is configured to:
determine VTA radius limits using the stimulation settings from the previous neurostimulation sessions; and
present a representation of the VTA radius limits relative to the anatomical structure on the user interface.

19. A non-transitory computer readable storage medium including instructions that when performed by a programming control circuit of a programming device of a neurostimulation system, cause the programming device to perform actions comprising:
receiving anatomical information associated with an anatomical structure of a patient;
retrieving stimulation settings from previous neurostimulation sessions, wherein the settings include at least one of stimulation location or stimulation amplitude;
identifying a volume of tissue activated (VTA) relative to the anatomical structure using the anatomical information and the stimulation settings;
determining a regression model relating the VTA to the anatomical structure; and
determining stimulation amplitude settings for the neurostimulation according to the regression model.

20. The non-transitory computer readable storage medium of claim 19, including instructions that cause the programming device to perform actions including:
determining a VTA radius for different amplitude settings and for different levels of a stimulation lead having electrodes at multiple levels; and
presenting, on a user interface of the programming device, a representation of the anatomical structure, the stimulation lead, and the VTA radius relative to the anatomical structure.
